(19) United States
(12) Patent Application Publication
(10) Pub. No.: US 2009/0048610 A1
(43) Pub. Date: Feb. 19, 2009

(54) MEDICAL PROBE INTRODUCER

(75) Inventors: Gideon Tolkowsky, Tev Aviv (IL); Dan Kinarty, Haifa (IL); Avishai Aizert, Kfar Saba (IL)

Correspondence Address: FOLEY & LARDNER LLP, 777 EAST WISCONSIN AVENUE, MILWAUKEE, WI, 53202-5306, US

(73) Assignee: BME Capital Holdings Ltd.

(21) Appl. No.: 11/838,759

(22) Filed: Aug. 14, 2007

(51) Int. Cl. A61B 19/00; A61B 5/04; A61M 5/178

(52) U.S. Cl. 606/130; 600/372; 604/164.11

(57) ABSTRACT

One embodiment of the invention relates to a system for placement of a cannula and an internal shaft into a patient including a base, a linear drive mechanism coupled to the base and a rotation mechanism coupled to the base. The base includes an attachment mechanism configured to be mechanically coupled to the patient to maximize placement accuracy. The system further includes a cannula coupled to the linear drive mechanism. The cannula has a longitudinal axis, a lumen, and a distal opening. The linear drive mechanism is configured to move the cannula in a linear direction along the longitudinal axis into the body and the rotation mechanism is configured to rotate the cannula about the longitudinal axis. The system further includes a shaft drive mechanism coupled to the linear drive mechanism and the rotation mechanism and a shaft slidably housed within the lumen of the cannula. The shaft drive mechanism is configured to move the shaft within the lumen of the cannula to deploy a distal tip of the shaft out of the distal opening of the cannula.

### BACKGROUND

With the proliferation of minimally invasive percutaneous medical procedures, there increasingly arises a need for placement of medical probes inside the human body at a high level of accuracy. This need is particularly, although not exclusively, apparent in neuro-diagnostic and neuro-therapeutic procedures, such as electrical neurostimulation, brain biopsy, brain tissue ablation, local drug delivery, and more. In some of these procedures, the requirement for high accuracy applies not only to the X, Y, and Z coordinates of the probe's distal tip inside the target tissue, but also to the angle of rotation of the probe relative to the tissue. Examples of such procedures include, but are not limited to the following types of medical probes and procedures:

1) A cannula that houses an internal elongated element whose distal tip is pre-bent sideways, where the pre-bent tip assumes a straight shape while it passes through the cannula and re-assumes its pre-bent shape when it emerges out of the cannula's distal tip, for delivery of a diagnostic or therapeutic procedure, such as electrical stimulation or local drug delivery or biopsy or ablation or brachytherapy or tissue monitoring with a sensor, to a tissue location that is off the cannula's longitudinal axis.

2) An optical fiber for delivery of photodynamic therapy, which is the light-based activation of light-sensitive chemotherapeutic drugs delivered to malignant tumors in order to kill the cancerous cells. Photo-dynamic therapy can be delivered to the target tissue by way of directing an optical fiber carrying a laser beam. Such direction requires both axial and rotational control if the target tissue is located off the probe's longitudinal axis.

3) An optical fiber for delivery of optical neurostimulation, which is an emerging application in the field of neurostimulation. Optical neurostimulation, which may partially replace electrical neurostimulation, involves light-based activation and de-activation of proteins embedded in neurons, which, when activated, trigger on and off neuronal electrical flashing. This can be a highly accurate method of stimulating individual neurons, in contrast to electrical neurostimulation that has a more diffused effect. Optical neurostimulation is direction-dependent, as a light beam is directional in nature. Optical neurostimulation may be done by way of directing an optical fiber to a specific tissue target. If the target tissue is located off the probe's longitudinal axis, such direction requires both axial and rotational control, at a high level of accuracy. Optical stimulation can also be applied to other types of cells, in addition to neurons, in order to trigger on specific cell activity, e.g., insulin release by pancreatic cells.

4) An optical fiber for delivering light to excite fluorescent nanoparticles in order to image tumor tissue during biopsies and surgeries. This emerging imaging technique can be particularly useful for precisely spotting a brain tumor during a surgery to remove the tumor, where patient outcome depends on successful removal of the entire tumor. In this imaging procedure, nanoparticles that emit infrared light when they are excited by visible light are injected into the tumor area and attach to malignant cells. An optical fiber then delivers light to the tumor area. The infrared rays emitted by the nanoparticles can be picked up by a small camera and viewed by the surgeon. The direction of an optical fiber to the tumor requires both axial and rotational control, at a high level of accuracy, if the target tissue is located off the probe's longitudinal axis.

One challenge associated with the high accuracy associated with minimally invasive percutaneous medical procedures is that certain surgical devices associated with introducing probes are configured such that the probe's coordinates coincide with room coordinates and can therefore present accuracy issues with respect to placement of the probe in the body. Further, the increasing use of robotic devices to perform minimally invasive diagnostic and therapeutic procedures presents a need for precision placement of tools in-vivo when utilizing robotic mechanisms.

It would be desirable to provide a system and/or method that satisfies one or more of these needs or provides other advantageous features. Other features and advantages will be made apparent from the present specification. The teachings disclosed extend to those embodiments that fall within the scope of the claims, regardless of whether they accomplish one or more of the aforementioned needs.

### SUMMARY

One embodiment of the invention relates to a system for placement of a cannula and an internal shaft into a patient. The system includes a base having an attachment mechanism configured to be mechanically coupled to the patient to fix the spatial position of the base relative to the patient to maximize placement accuracy. The system further includes a linear drive mechanism coupled to the base, and a rotation mechanism coupled to the base. The system further includes a cannula coupled to the linear drive mechanism. The cannula has a longitudinal axis, a lumen, and a distal opening. The linear drive mechanism is configured to move the cannula in a linear direction along the longitudinal axis into the body and the rotation mechanism is configured to rotate the cannula about the longitudinal axis. The system further includes a shaft drive mechanism coupled to the linear drive mechanism and the rotation mechanism and a shaft slidably housed within the lumen of the cannula. The shaft drive mechanism is configured to move the shaft longitudinally but not rotationally within the lumen of the cannula to deploy a distal tip of the shaft out of the distal opening of the cannula.

Another embodiment of the invention relates to a method of diagnosing or providing a medical treatment to a target tissue of a patient using the probe introducer system described above. The method includes coupling the base of the system to the patient, creating an aperture in the patient sized to receive the cannula, advancing the cannula into the aperture with the linear drive mechanism until the distal opening of the cannula is located proximate the target tissue, rotating the cannula with the rotation mechanism to a desired angle, deploying the distal tip of the shaft out of the distal opening of the cannula with the shaft drive mechanism, and diagnosing or providing a medical treatment to the target tissue. The medical treatment may include delivering a therapeutic liquid, draining a liquid, performing electrical or optical stimulation of neurons and other cells, performing a biopsy, delivering a brachytherapy seed, performing photodynamic therapy, performing tissue ablation, or performing tissue diagnosis or monitoring. The base of the system may be coupled to the patient by way of direct fixation to the outside surface of the body or by fixation to an intermediate structure such as a stereotactic frame.

The invention is capable of other embodiments and of being practiced or being carried out in various ways. Alternative exemplary embodiments relate to other features and combinations of features as may be generally recited in the claims.

### BRIEF DESCRIPTION OF THE DRAWINGS

The invention will become more fully understood from the following detailed description, taken in conjunction with the accompanying drawings, wherein like reference numerals refer to like elements, in which:

FIG. 1 is an isometric view of a medical probe introducer coupled to a patient according to an exemplary embodiment.

FIG. 2 is an isometric view of a medical probe introducer coupled to a patient with a stereotactic frame according to an exemplary embodiment.

FIG. 3 is an perspective view of the medical probe introducer of FIG. 1 according to an exemplary embodiment.

FIG. 4 is a cross-section of the base of the medical probe introducer of FIG. 2 taken along line 4-4.

FIG. 5 is an perspective view of a portion of the medical probe introducer of FIG. 2 showing the indicator for the rotation mechanism.

FIGS. 6A and 6B are perspective views of a portion of the medical probe introducer of FIG. 2 showing the linear drive mechanism.

FIG. 7 is an perspective view of a portion of the medical probe introducer of FIG. 2 showing the shaft drive mechanism.

FIG. 8 is a cross-section of a portion of the shaft drive mechanism of FIG. 7 taken along line 8-8.

FIG. 9 is a cross section of a portion of a shaft mechanism according to another exemplary embodiment including a shaft screw with a central bore configured to receive the shaft.

FIGS. 10-12 are partial cross-section views of a portion of a patient's body showing a pre-bent shaft being deployed from a cannula proximate to the target tissue.

FIGS. 13-14 are partial cross-section views of a portion of a patient's body showing a shaft including an optical fiber.

FIG. 15 is a flowchart showing a method of providing treatment to a target tissue of a patient according to an exemplary embodiment.

### DETAILED DESCRIPTION OF THE PREFERRED EMBODIMENTS

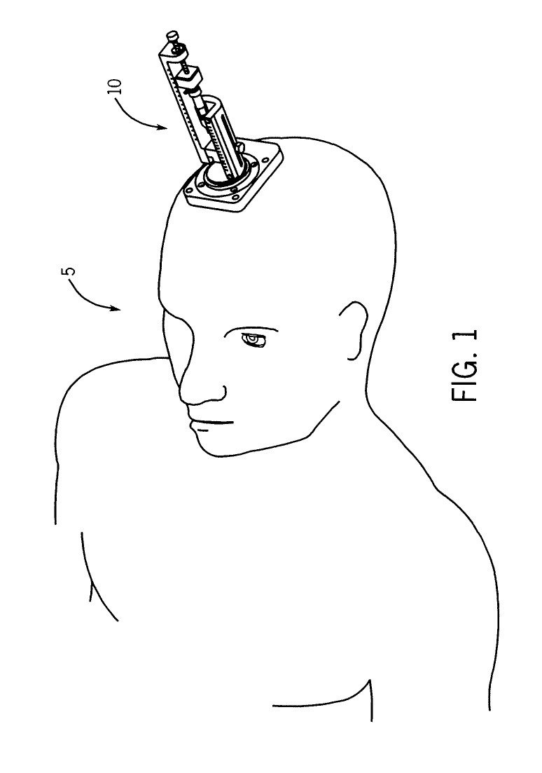

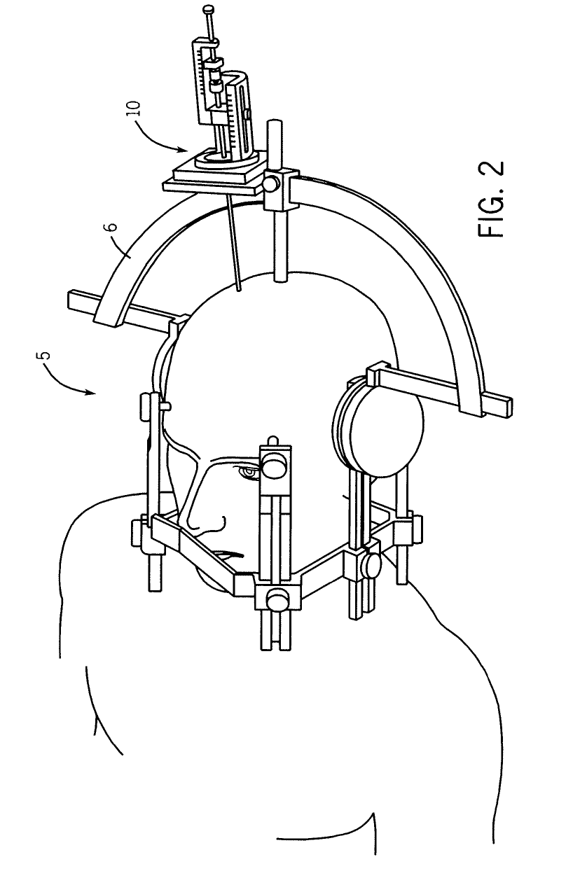

Referring in general to the FIGURES, a medical probe introducer 10 is shown according to an exemplary embodiment. The medical probe introducer 10 is configured to allow a medical probe to be introduced into the body of a patient 5 such that the X-Y-Z position of the tip of the probe, as well as the rotational orientation of the probe, may be controlled with micrometer precision. Such precision is desirable to direct a medical probe to an objective tissue in the patient's body with minimal damage or disturbance to the surrounding tissue, especially in diagnostic and therapeutic procedures conducted in the central nervous system. The target tissue includes but is not limited to brain or other neural tissue that needs to be stimulated electrically or optically, or a malignant tumor to which a chemotherapeutic drug is to be locally delivered, or a suspected tumor that needs to be sampled by way of biopsy, or a malignant tumor that needs to be ablated or locally radiated, or pathological tissue such as ischemic tissue in the brain that needs to be monitored by a biochemical or physical sensor. The medical probe introducer 10 may be coupled directly to the patient as shown in FIG. 1 or may be coupled to an intermediate structure such as a stereotactic frame 6 as shown in FIG. 2.

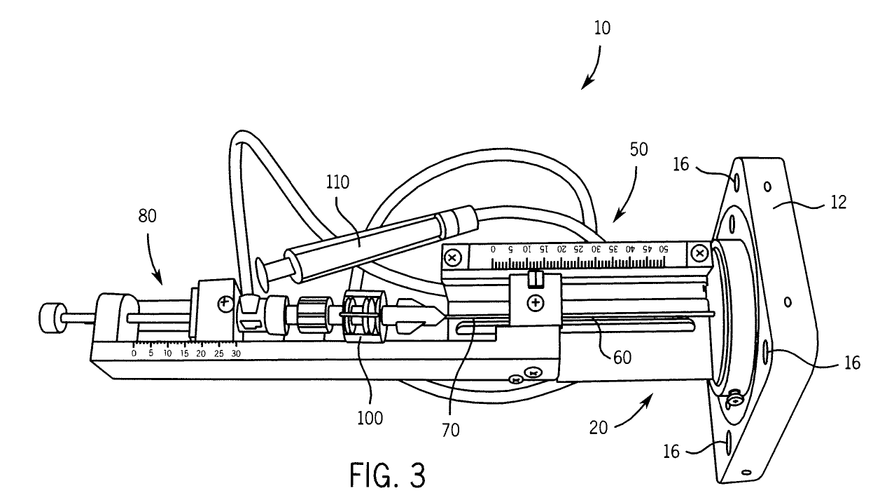

Referring now to FIG. 3, the medical probe introducer 10 includes a base 12, a rotation mechanism 20 and a linear drive mechanism 50. An outside needle or cannula 60 is coupled to the rotation mechanism 20 and the linear drive mechanism 50. The cannula 60 is an elongated member with a central longitudinal axis 62. The linear drive mechanism 50 moves the cannula 60 along the axis 62 while the rotation mechanism 20 rotates the cannula 60 about the axis 62. A shaft drive mechanism 80 is coupled to the linear drive mechanism 50. An inside needle or shaft 70 is housed within the cannula 60 and is moved along the central longitudinal axis 62 by the shaft drive mechanism 80. The shaft 70 may be configured for a wide variety of medical procedures and may be a variety of mechanisms including, but not limited to: an optical fiber for optical stimulation or neurons or other cells or for photodynamic therapy, a needle, a shunt, an electrical stimulation lead, a neurostimulation electrode, a biopsy needle, an ablation catheter, or a diagnostic sensor. The shaft 70 may be configured to deliver or remove a liquid and may be coupled to a liquid delivery device shown as a syringe 110 with a connector 100.

In an exemplary embodiment, the base 12 (e.g., static base) is a plate-like member to which the other components of the system are coupled. The base 12 includes a central opening that receives the rotation mechanism 20. The base 12 further includes an attachment mechanism, shown as multitude of openings 16 that receive fasteners to couple the base to the skull of a patient (e.g., with surgical screws). In another embodiment, the attachment mechanism is a stereotactic frame 6 coupled to the patient. The base may be coupled to the patient or to the stereotactic frame 6 with an adhesive or with another suitable mechanism. The base 12 is formed from a biocompatible solid material such as stainless steel (e.g., SST 303).

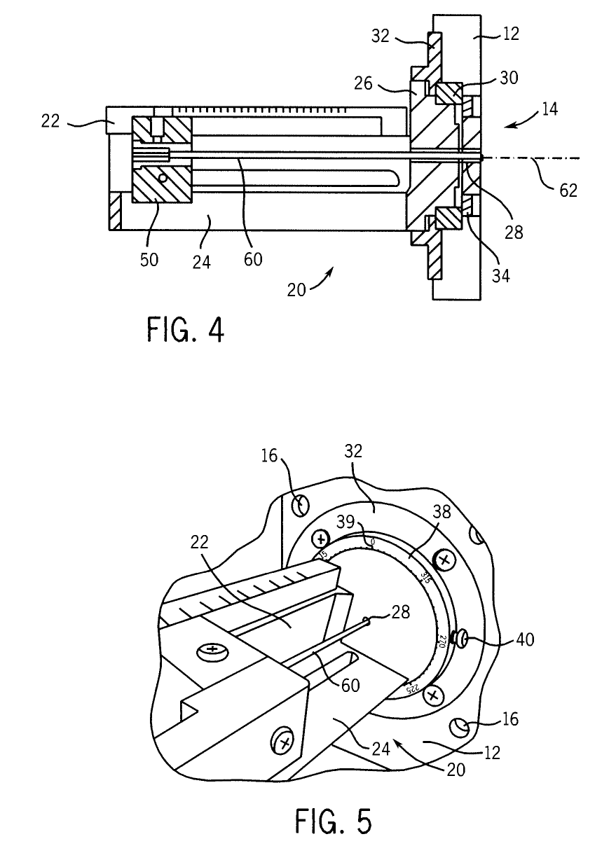

The rotation mechanism 20 (e.g., common dynamic base) is coupled to the base 12 and allows the cannula 60 and the shaft 70 to be rotated about the central longitudinal axis 62. According to one exemplary embodiment, the rotation mechanism 20 includes a main body with a generally L-shaped profile formed by a first wall 22 and a second wall 24. The main body of the rotation mechanism 20 further includes a circular end wall 26 that is perpendicular to the first wall 22 and the second wall 24. As shown best in FIG. 4, the end wall 26 is received in the central opening 14 of the base 12 and includes an aperture 28 that is aligned with an opening 7 (see FIGS. 9-11) in the patient so that the cannula 60 may pass through the aperture 28 and the opening 7. According to an exemplary embodiment, the main body of the rotation mechanism 20 is formed from a relatively light weight solid material such as aluminum (e.g., Al 6061).

The rotation mechanism 20 is coupled to the base 12 with a bearing assembly including a bearing 30, a lock plate 32 and an inside bearing nut 34. In this way, the rotation mechanism 20 is linearly fixed to the base 12 but may still rotate relative to the base 12 about the axis 62. As shown best in FIG. 5, a rotational scale 38 with angular indicators including a zero or datum indicator 39 is provided to show the relative rotational position of the rotation mechanism 20 relative to the base 12. According to an exemplary embodiment, the rotational scale 38 includes indicators from 0-360 degrees at 5 degree intervals. According to other exemplary embodiments, the rotational scale 38 may have a different scale (e.g., radians, etc.) and may have more or fewer indicators. A set screw 40 is used to selectively fix the rotation mechanism 20 relative to the base 12. To rotate the rotation mechanism 20 relative to the base 12, the set screw 40 is loosened. When the set screw 40 is tightened, is contacts the rotation mechanism 20 and effectively fixes the rotation mechanism 20 so that it may not rotate relative to the base 12. The rotation of the rotation mechanism 20 may be controlled manually, robotically through a gear system (e.g. by a robotic mechanism), by a screw, or by a micrometer.

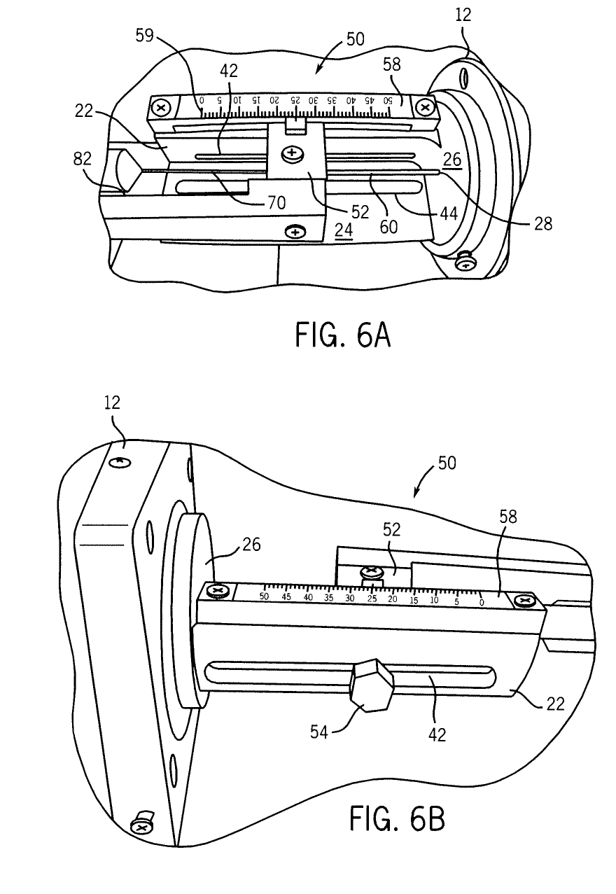

Referring now to FIGS. 6A and 6B, the linear drive mechanism 50 is shown. The linear drive mechanism 50 (e.g., linear dynamic base) is coupled to the rotation mechanism 20 and allows the cannula 60 and the shaft 70 to be moved along the central longitudinal axis 62 into and out of the patient 5. The linear drive mechanism 50 includes a main body 52 (e.g., clamp, connector, bracket, etc.) that is coupled to the cannula 60 such that the cannula is aligned with the aperture 28 in the rotation mechanism 20. The body 52 is coupled to the rotation mechanism 20 with a bolt 54. The bolt 54 extends through a slot 42 in the first wall 22 of the rotation mechanism and engages the body 52. When it is loosened, the bolt 54 slides along the slot 42 and allows the body 52 to move relative to the rotation mechanism 20. When it is tightened, the bolt 54 forces the body 52 against the first wall 22 and effectively fixes the body 52 so that it may not move relative to the rotation mechanism 20. According to one exemplary embodiment, the second wall 24 includes a slot 44 through which passes a second bolt (not shown). The second bolt engages the body 52 and cooperates with the first bolt 54 to fix the body 52 relative to the rotation mechanism 20. Each of the bolts may be controlled manually, robotically through a gear system (e.g. by a robotic mechanism), by a screw, or by a micrometer.

The linear drive mechanism 50 includes a linear scale 58. The linear scale 58 includes a multitude of indicators including a zero or datum indicator 59 to show the relative linear movement of the linear drive mechanism 50 relative to the rotation mechanism 20 and the base 12. According to an exemplary embodiment, the linear scale 58 includes indicators from 0-50 mm at 1 mm increments. According to other exemplary embodiments, the linear scale may have a different scale (e.g., inches, etc.) and may have more or fewer indicators. The movement of the linear drive mechanism 50 may be controlled manually, robotically through a gear system (e.g. by a robotic mechanism), by a screw, or by a micrometer.

The body 52 is coupled to the cannula 60 (e.g., rigid needle, outside needle, insertion needle, etc.). The cannula 60 is an elongated tube-like member that is formed from a biocompatible solid material such as stainless steel (e.g., SST 303) and is configured to be inserted into the patient 5 as shown in FIGS. 9-11. The cannula 60 has an inner diameter that forms a generally tubular cavity or lumen 64 that is configured to allow the shaft 70 to slide within the cannula 60. According to an exemplary embodiment, the inner diameter of the cannula 60 is approximately 10% larger than the outer diameter of the shaft 70. The outer diameter of the cannula 60 is large enough to allow the cannula to be strong enough to overcome the resistance of the tissue into which it will be inserted (e.g., to prevent buckling). According to an exemplary embodiment, the outside diameter of the cannula 60 is less than 1.27 mm. The cannula 60 is coupled to the body 52 and is aligned with the aperture 28 in the rotation mechanism 20. The cannula 60 is moved linearly along the longitudinal axis 62 (e.g., in and out) by the linear drive mechanism 50 and the cannula 60 is rotated about the longitudinal axis 62 by the rotation mechanism 20.

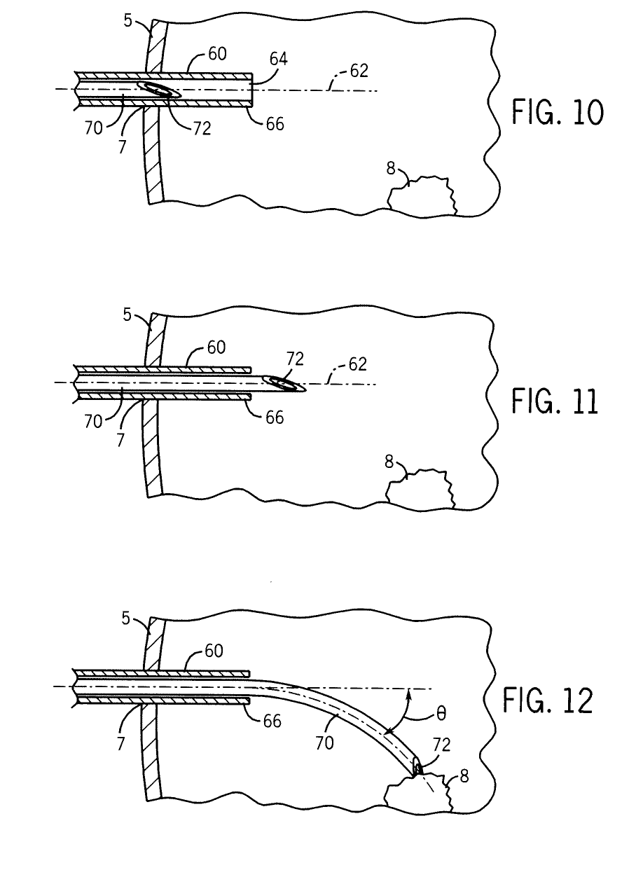

The shaft 70 is an elongated element that that is configured to be deployed from the cannula 60. The shaft 70 is at least partially nested within the cannula 60 and has a diameter smaller than the diameter of the lumen 64 of the cannula 60. As shown best in FIGS. 10-12, the distal end or segment 72 of the shaft 70 is configured to extend beyond the distal end 66 of the cannula 60 to interact with the target tissue 8 in the patient 5. According to one exemplary embodiment, the shaft 70 is between approximately 10% and 50% longer than the cannula 60. The shaft 70 formed from a biocompatible solid material such as stainless steel or nitinol (nickel-titanium alloy). According to various exemplary embodiments, the shaft 70 may be an injection needle, a syringe, a drainage shunt, an electrode, an optical fiber for therapy delivery or for tissue viewing, a biopsy needle, an ablation catheter, a brachytherapy catheter, a catheter carrying a biochemical or a physical sensor at its tip, or any other diagnostic or therapeutic medical device that is used percutaneously.

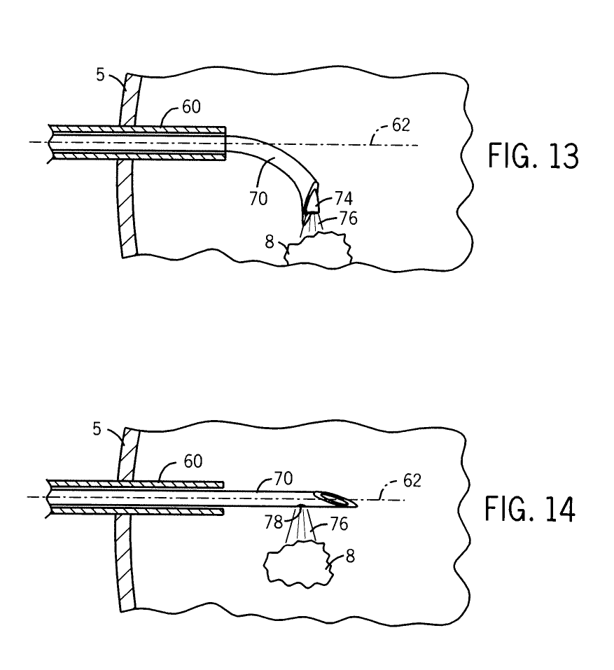

According to some exemplary embodiments, distal segment 72 of the shaft 70 can be manufactured in such way that it is bent (e.g., in a 90 degree arc). In such exemplary embodiments, the distal segment 72 assumes a straight shape (e.g., aligned with the longitudinal axis 62) while it passes through the cannula 60 (as shown in FIG. 10). As it emerges beyond the distal end 66 of the cannula 60, the distal segment 72 re-assumes its bent shape and is oriented at an angle θ relative to the longitudinal axis 62 (as shown in FIG. 11). According to another embodiment, the shaft 70 may contain an optical fiber 74 that projects a light beam 76 sideways relative to the longitudinal axis 62, either by bending relative to the longitudinal axis 62 (as shown in FIG. 13) or by projecting the light through a side opening 78 in the shaft that serves as a window (as shown in FIG. 14).

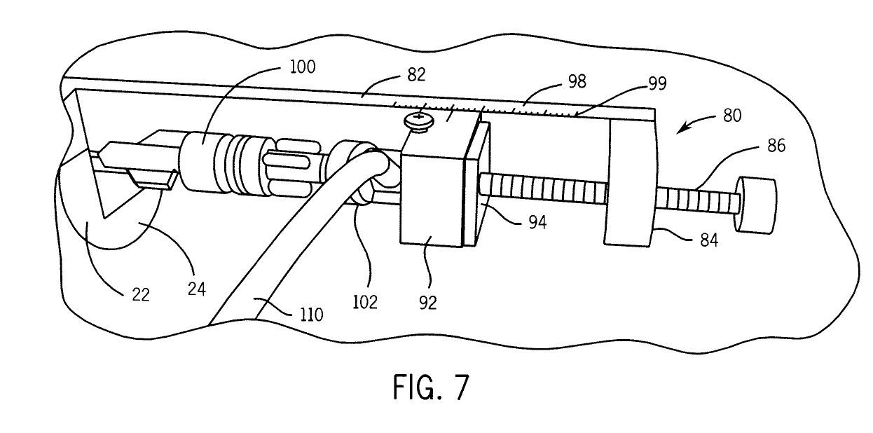

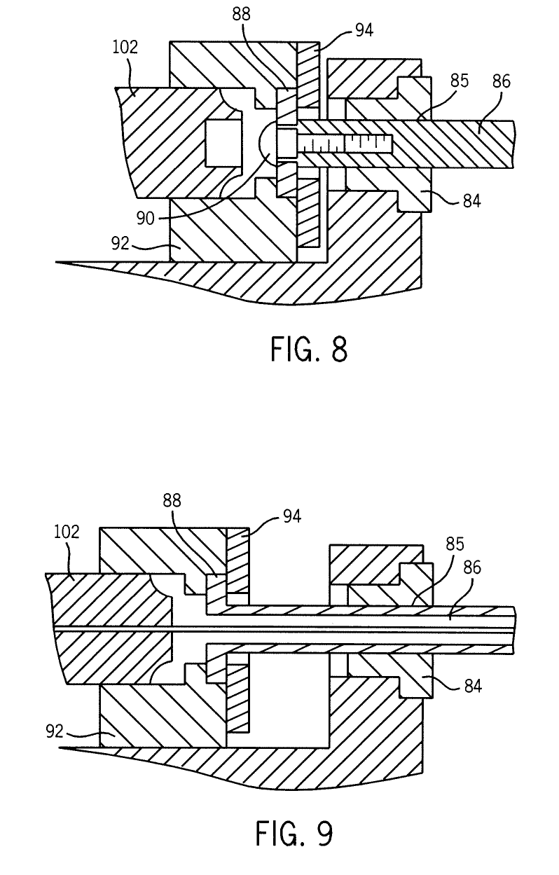

Referring especially to FIGS. 7-8, the shaft drive mechanism 80 is shown. The movement of the shaft 70 relative to the cannula 60 is controlled by the shaft drive mechanism 80. The shaft drive mechanism 80 includes a bracket or rail 82 coupled to the body 52, a back thread adapter 84, a shaft screw 86, a disc 88, a screw 90, an adaptor 92, and a cover plate 94. The rail 82 extends backwards from the body 52 away from the base 12. The back thread adaptor 84 is coupled to the rail 82 opposite of the body 52. The back thread adaptor 84 includes a threaded opening 85 that receives the shaft screw 86. As the shaft screw 86 is turned, the end of the shaft screw 86 advances towards the base 12 or retreats away from the base 12. A disc 88 is coupled to the end of the shaft screw 86 with a fastener, shown as screw 90. The disc 88 is trapped between the adaptor 92 and the cover plate 94 coupled to the adaptor 92. In this way, the disc 88 causes the adaptor 92 to move with the shaft screw 86 as the shaft screw 86 is rotated. According to an exemplary embodiment, the disc 88 is formed from a material with a low coefficient of friction (e.g., Teflon® brand non-stick coating) to reduce the chance of the disc 88 locking up or binding with the adaptor 92 and or the cover plate 94 when the shaft screw 86 is turned. According to various exemplary embodiments, the shaft screw 86 may have a simple screw head or may have a micrometer head. Referring to FIG. 9, according to embodiments where the shaft is used for a medical procedure other than the injection or drainage of liquids (e.g., a biopsy, electrical stimulation, optical stimulation, ablation, etc.), the shaft screw 86 may include a centric hole (e.g., lumen, bore, etc.) through which the shaft 70 is inserted into the cannula 60 and then fastened to the cannula 60. The shaft screw 86 may be controlled manually, robotically through a gear system (e.g. by a robotic mechanism), or by a micrometer.

The shaft drive mechanism 80 includes a linear scale 98. The linear scale 98 includes a multitude of indicators including a zero or datum indicator 99 to show the relative linear movement of the shaft drive mechanism 80 relative to the linear drive mechanism 50. According to an exemplary embodiment, the linear scale 98 includes indicators from 0-30 mm at 1 mm increments. According to other exemplary embodiments, the linear scale may have a different scale (e.g., inches, etc.) and may have more or fewer indicators.

The adaptor 92 is coupled to the shaft 70 with connectors 100. The connectors may be a commonly known connector such as a Luer connector. The medical probe introducer 10 may also include a Y-type connector 102 that allows a the connectors 100 and the shaft 70 to be in fluid communication with a liquid delivery device 110 shown in FIG. 3 as a syringe and tube to facilitate the injection or draining of fluid.

The rotation mechanism 20, the linear drive mechanism 50, and the shaft drive mechanism 80 cooperate to allow a probe to be introduced into the patient with a high degree of axial and angular accuracy. The mechanisms 20, 50, and 80 may be used to control the axial position of the cannula 60, the axial position of the shaft 70, and the angular position of the cannula 60 and the shaft 70 at sub-millimeter or sub-degree accuracy. The mechanisms 20, 50, and 80 may be controlled either manually or robotically. The controller can exchange data with an imaging device, such as ultrasound or CT or MRI. If controlled robotically by a robotic mechanism, the mechanisms 20, 50, and 80 may be controlled via gears or other intermediate devices by a computerized controller and interface with the imaging device in real time, thus enabling image-guided placement of the shaft precisely at the desired tissue location. The medical probe introducer 10 may also be used in conjunction with a system for mapping the target tissue area, e.g., electrical mapping of brain tissue, to maximize accuracy of delivery.

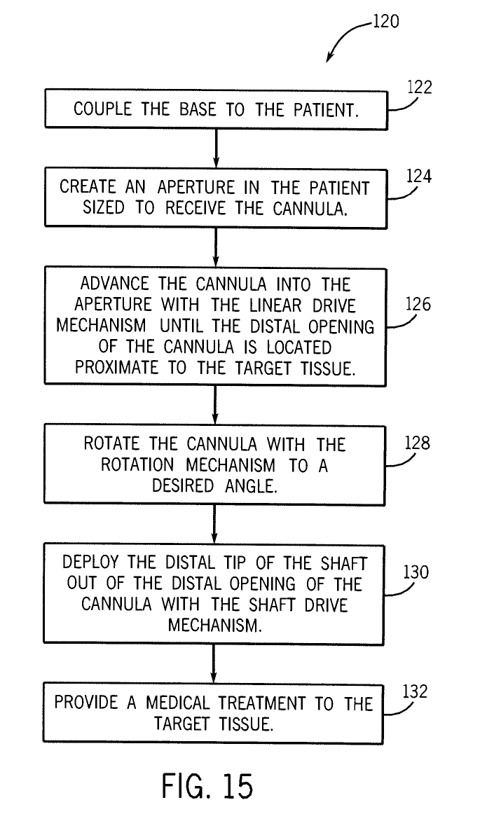

Referring to FIG. 15, a method of providing a medical treatment to a target tissue 120 is described according to an exemplary embodiment. In a first step, the base 12 is coupled to a stereotactic frame 6 attached to the patient's body 5 (e.g., to the skull) or directly to the skull by way of surgical screws (step 122). An opening or aperture 7 is then created to allow the cannula 60 to be inserted into the body 5 (step 124). For example, if the procedure is performed on the brain, a burr hole is drilled in the skull. The cannula 60 is percutaneously advanced into the patient's body 5 with the linear drive mechanism 50 until the distal tip 66 of the cannula 60 is positioned proximate to the target tissue 8 (step 126). The rotation mechanism 20 is then rotated to an angle such that the shaft 70, once deployed out of the cannula 60, will be positioned to deliver the diagnostic or therapeutic procedure to the target tissue 8 (step 128). The shaft drive mechanism 80 is then advanced to position the distal tip 72 of the shaft 70 proximate to the target tissue 8 (step 130). The medical treatment is then provided to the target tissue 8 (step 122). If the medical treatment includes delivering or draining diagnostic or therapeutic liquids to the target tissue 8, then a Y-connector 102 and connectors 100 are used to couple a liquid delivery device 110 to a hollow needle that serves as the shaft 70. If the procedure involves accurately placing a medical tool (e.g., a neurostimulation electrode or optical fiber, a biopsy needle, an ablation catheter, a diagnostic sensor, etc.) then the tool serves as the shaft 70 and is inserted into the cannula 60 through a lumen in the center of the shaft screw 86 and fastened to the cannula 60. Once the procedure is completed, the shaft 70 is retracted into the cannula 60 by turning the shaft screw 86, the cannula 60 is retracted with the linear drive mechanism 50, and the base 12 is detached from the stereotactic frame 6 or from the patient's body.

As can be appreciated by those skilled in the art, the medical probe introducer as described herein may have a wide variety of applications. According to one exemplary embodiment, the introducer may be used to direct an electrical or optical stimulation lead or fiber towards brain tissue to electrically or optically stimulate the brain tissue (e.g., to treat Parkinson's or Epilepsy, etc.). According to another exemplary embodiment, the introducer may be used to precisely install a medicine or contradiction fluid for medical imaging. According to another exemplary embodiment, the introducer may be used to locally burn cancerous tissue. According to another exemplary embodiment, the introducer may be used to remove a portion of tissue such as for a biopsy.

The described configuration of the medical probe introducer having an attachment mechanism permitting the introducer to be mechanically coupled to the patient addresses the accuracy challenge presented by minimally invasive percutaneous medical procedures. Because the introducer may be firmly attached (either directly or indirectly via an intermediate frame) to the treated patient body part (e.g. skull) thus having the probe's and the body part's spatial geometric coordinates coincide with no relative movement between them, a high degree of accuracy may be achieved, in contrast to devices in which the probe's coordinates coincide with room coordinates.

The construction and arrangement of the elements of the medical probe introducer as shown in the various exemplary embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited herein. For example, elements shown as integrally formed may be constructed of multiple parts or elements, the position of elements may be reversed or otherwise varied, and the nature or number of discrete elements or positions may be altered or varied. It should be noted that the elements and/or assemblies of the system may be constructed from any of a wide variety of materials that provide sufficient strength, durability, or biocompatibility. Other substitutions, modifications, changes and omissions may be made in the design, operating conditions and arrangement of the preferred and other exemplary embodiments and medical procedures without departing from the scope of the present invention.

What is claimed is:

1. A system for placement of a cannula and an internal shaft into a patient, comprising:
a base including an attachment mechanism configured to be mechanically coupled to the patient to fix the spatial position of the base relative to the patient;
a linear drive mechanism coupled to the base;
a rotation mechanism coupled to the base;
a cannula coupled to the linear drive mechanism, the cannula having a longitudinal axis, a lumen, and a distal opening, wherein the linear drive mechanism is configured to move the cannula in a linear direction along the longitudinal axis into the body and wherein the rotation mechanism is configured to rotate the cannula about the longitudinal axis;
a shaft drive mechanism coupled to the linear drive mechanism and the rotation mechanism; and
a shaft slidably housed within the lumen of the cannula, wherein the shaft drive mechanism is configured to longitudinally but not rotationally move the shaft within the lumen of the cannula to deploy a distal tip of the shaft out of the distal opening of the cannula.

2. The system of claim 1, wherein the shaft drive mechanism is configured relative to both the linear drive mechanism and the rotation mechanism such that the shaft moves in a linear direction with the cannula and rotates with the cannula.

3. The system of claim 1, wherein at least one of the linear drive mechanism and the shaft drive mechanism provides for micrometer precision in the linear adjustment of the shaft and/or the cannula.

4. The system of claim 1, wherein the rotation mechanism provides for micrometer precision in the rotation of the cannula and the shaft.

5. The system of claim 1, wherein the shaft is at least one of an optical fiber, a needle, a shunt, and an electrical stimulation lead.

6. The system of claim 1, wherein the shaft is at least one of a neurostimulation electrode, a neurostimulation optical fiber, an optical fiber for delivering photodynamic therapy, a biopsy needle, an ablation catheter, a drainage catheter, a needle for the delivery of a drug or diagnostic agent, and a diagnostic sensor.

7. The system of claim 1, wherein the distal tip of the shaft has a pre-bent shape, wherein the distal tip maintains a straight configuration when within the lumen and assumes the pre-bent shape when deployed out of the lumen.

8. The system of claim 1, wherein the shaft is cannulated and includes a proximal connection point for a liquid delivery device.

9. The system of claim 1, further comprising a computerized controller configured to control at least one of the linear drive mechanism, the shaft drive mechanism, and the rotation mechanism.

10. The system of claim 1, further comprising an imaging system configured to provide an image of the body to aid in placement of at least one of the cannula and the shaft within the body.

11. The system of claim 1, wherein the shaft drive mechanism is mounted on the linear drive mechanism and comprises a shaft screw.

12. The system of claim 11, wherein the shaft screw comprises a central lumen configured to receive the shaft, wherein the shaft is configured to be fastened to the shaft screw.

13. The system of claim 1, further comprising a robotic mechanism configured to control at least one of the rotation mechanism, the linear drive mechanism, and the shaft drive mechanism to move at least one of the cannula and the shaft.

14. The system of claim 1, wherein the cannula is rigid.

15. The system of claim 1, wherein the attachment mechanism includes a plurality of apertures configured to receive surgical screws for coupling the base to the patient.

16. A method of diagnosing or providing a medical treatment to a target tissue of a patient using the system of claim 1, comprising:
coupling the base to the patient;
creating an aperture in the patient sized to receive the cannula;
advancing the cannula into the aperture with the linear drive mechanism until the distal opening of the cannula is located proximate the target tissue;
rotating the cannula with the rotation mechanism to a desired angle;
deploying the distal tip of the shaft out of the distal opening of the cannula with the shaft drive mechanism; and
diagnosing or providing a medical treatment to the target tissue.

17. The method of claim 16, wherein the medical treatment is at least one of delivering a therapeutic liquid, draining a liquid, performing electrical or optical stimulation of neurons or other cells, performing a biopsy, delivering a brachytherapy seed, performing photodynamic therapy, performing tissue ablation, and performing tissue diagnosis or monitoring.

18. The method of claim 16, wherein the coupling step comprises using the attachment mechanism to couple the base to the patient using surgical screws.

19. The method of claim 16, wherein the base is coupled to the patient indirectly via a stereotactic frame.

20. The method of claim 16, wherein the shaft comprises an optical fiber and wherein the medical treatment comprises delivering light to excite fluorescent nanoparticles to image tumor tissue.

21. The method of claim 16, wherein the advancing, rotating, and deploying steps are performed manually by a user.

22. The method of claim 16, further comprising providing a robotic mechanism and moving at least one of the cannula and shaft by controlling at least one of the rotation mechanism, the linear drive mechanism, and the shaft drive mechanism with the robotic mechanism.

23. The method of claim 16, further comprising providing an imaging device and exchanging data between the robotic mechanism and the imaging device.
